(19) United States
(12) Patent Application Publication
(10) Pub. No.: US 2022/0379117 A1
(43) Pub. Date: Dec. 1, 2022

(54) SYSTEMS AND METHODS FOR MONITORING BIOELECTRICAL ACTIVITY AND ASSESSING CONDITIONS ASSOCIATED THEREWITH

(71) Applicant: CoreMap, Inc., Colchester, VT (US)

(72) Inventor: Peter S. Spector, Colchester, VT (US)

(21) Appl. No.: 17/751,844

(22) Filed: May 24, 2022

Related U.S. Application Data
(60) Provisional application No. 63/192,949, filed on May 25, 2021.

(51) Int. Cl. A61N 1/36

(52) U.S. Cl. CPC A61N 1/36135; A61N 1/3606

(57) ABSTRACT

The invention provides systems and methods for monitoring electrical biosignals of one or more biological cells, tissues, and/or organs of a patient and assessing a condition of the patient based on the electrical biosignals.

### CROSS-REFERENCE TO RELATED APPLICATION

This application claims priority to, and the benefit of, U.S. Provisional Application No. 63/192,949, filed on May 25, 2021, the content of which is incorporated by reference herein in its entirety.

### TECHNICAL FIELD

The invention generally relates to diagnostic systems and methods, and, more particularly, to systems and methods for monitoring electrical biosignals of one or more biological cells, tissues, and/or organs and assessing a condition based on the electrical biosignals.

### BACKGROUND

Electrophysiological signals are useful in medical diagnostics. Electrophysiological signals originate from the muscular, cardiac or neurological activity. Electrical biosignals (also referred to herein as “bioelectrical signals”) are low amplitude and low frequency electrical signals that can be measured through changes in electrical potential across a cell, tissue, or organ. Bioelectrical signals include measurements of biopotentials and bioimpedance. Biopotentials reflect electrical activity in tissue, while bioimpedance measurements include electric currents that are supplied from an external source outside living tissues. Accordingly, biopotentials generally refer to active processes, such as excitation of nerve and muscle tissues, whereas bioimpedance is related to passive properties of the tissue, such as the properties of the skin.

The most common bioelectrical signals include, but are not limited to, electroencephalogram (EEG), electrocardiogram (ECG), electromyogram (EMG), electrooculogram (EOG), electroretinogram (ERG), electrogastrogram (EGG), and galvanic skin response (GSR) or electrodermal activity (EDA).

While conventional technologies allow for monitoring and evaluating bioelectrical activity, there are drawbacks. For example, in order to measure a bioelectrical signal, a patient is generally equipped with electrodes for sensing electrical activity at a target site or an area of body generally associated with the cell, tissue, or organ of interest (i.e., heart, brain, skeletal muscle, etc.). The measured signal is based on the potential between the electrodes, which is dependent on the sum of the neural and muscular electronic activity between the electrodes. The quality of the signal is greatly affected by the accuracy of the position of the electrode, as well as the conductance of a given surface upon which the electrodes are positioned.

Existing electrode configurations used for monitoring bioelectrical activity have several shortcomings, including the electrodes being too large, the inter-electrode spacing is too great, and the electrode configurations are not suitably orthogonal to the tissue surface. Such configurations of require time consuming methods since the monitoring device (e.g., typically a catheter and electrodes) has to be moved to a relatively large number of locations associated with target site to acquire sufficient data. Additionally, moving the device to different locations so that the electrodes touch the desired tissue is a cumbersome process that is technically challenging.

### SUMMARY

The present invention provides systems and methods for monitoring bioelectrical signals in cells, tissues, and/or organs and assessing a health condition based on the bioelectrical signals. The invention makes use of a unique and innovative electrode design.

Electrode design in the present invention takes the form of an array of single electrodes or an electrode array comprising a plurality of stacked electrode pairs. Either embodiment of the invention produces results provided herein and will be described separately herein.

In one embodiment of the invention, an electrode pair comprises a first electrode configured to be in contact with a surface of a desired target site (i.e., cells, tissue, organ or the like from which bioelectrical signals are to be recorded) and a second electrode separated from the first electrode. Each electrode pair is generally arranged in an orthogonal, close, unipolar (OCU) configuration. More specifically, the common axis between the first and second electrodes (referred to as the “inter-electrode axis”) is “orthogonal” to a given surface at the target site when a recording is performed. The distance between the first and second electrodes (referred to as “inter-electrode distance”) is substantially “close”, which may be within an order of magnate of the electrode size (i.e., from 0.1 mm to 3.0) or greater, such that the second electrode is “close” enough to the surface of the target site to detect a bioelectrical signal. The electrode pair may be “unipolar” in that only the first electrode may be in contact with the surface of the target site.

Such a configuration addresses the limitations of conventional unipolar and bipolar electrodes. In particular, recorded electrical potential of current bipolar electrodes vary with their orientation relative to the direction of a passing wavefront. Additionally, because bipolar electrodes have both electrodes on a given surface, there is potential inclusion of distinctly different electrical activity from each electrode. As such, by providing electrodes oriented perpendicular to the tissue plane (i.e., via the orthogonal close unipolar (OCU) design of the present invention), the two-dimensional electrode array of the present disclosure retains the superior near/far-field discrimination of common bipolar electrode recordings with the directional independence and smaller footprint of unipolar recordings. Furthermore, the unipolar electrode configuration of the present invention retains all of the spatial resolution benefits of a contact bipolar configuration, but with the additional spatial resolution enhancement conferred by a smaller footprint (i.e., only half of the electrodes, the first electrode of each electrode pair, may be in contact with the tissue surface).

Accordingly, the spatial configuration of the electrode array (i.e., the OCU design) provides the benefits of existing unipolar and bipolar electrode designs without the drawbacks, all while providing high spatial resolution, thereby improving bioelectrical signal sensing. As such, the present invention is suitable for numerous applications in which bioelectrical activity monitoring is required and high resolution is desired, and is not limited to use in any given field.

For example, an electrode array configuration of the present invention is useful for monitoring activity for a specific organ or system (i.e., heart, brain, nervous system, or the like), and such data collected can be used for assessing a condition (i.e., physiological, psychological, cognitive, etc.). By monitoring bioelectrical activity of a given target site, the collected signal data can be used in diagnosing a patient with a condition, including any known disorders, simply based on an analysis of the signal data.

Additionally, the collection of bioelectrical activity data can be used in other applications and is not limited to simply diagnosis a patient with a particular condition or disorder. In particular, the present invention is useful in a research setting, including drug screening applications for testing the safety and efficacy of a drug on a subject (i.e., animal studies). For example, depending on the particular condition undergoing testing, the electrode array may be useful in monitoring bioelectrical activity of a given target site (i.e., monitoring brain activity and/or nervous tissue of the central nervous system for Parkinson's disease research) before and after application of a drug, in which such bioelectrical signal data may be useful in determining the effectiveness, as well as any side effects, of the drug. Furthermore, the present invention is useful in providing real-time bioelectrical activity feedback of a given target site during a treatment procedure in which the target site is subjected to a treatment (i.e., application of energy or electrical stimulation), to thereby provide a medical professional with feedback of the effectiveness (or ineffectiveness) of the treatment based on the measured bioelectrical signal data.

In one aspect, the present invention includes a method for assessing a condition of a patient. The method comprises positioning a two-dimensional electrode array at a target site. The electrode array comprises a plurality of stacked electrode pairs each comprising a first electrode configured to be in contact with a surface of the target site and a second electrode separated from the first electrode, wherein one or more the electrode pairs are arranged orthogonal to the surface of the target site. The method further includes sensing bioelectrical signals via one or more of the electrode pairs and obtaining electrophysiological measurements from the bioelectrical signals in response to bioelectrical activity associated with the target site. The method further includes assessing a condition of the patient based, at least in part on, the electrophysiological measurements.

The condition may include at least one of a physiological disorder, psychological disorder, and cognitive disorder. The condition may include, for example, Alzheimer's disease, Parkinson's disease, other types of movement disorders, seizure disorders (e.g., epilepsy), urinary or fecal incontinence, sexual dysfunction, obesity, mood disorders, gastroparesis, or diabetes.

The first electrode of each stacked electrode pair is configured to record a first bioelectrical signal and the second electrode of each stacked electrode pair is separated from the first electrode by a distance which enables the second electrode to record a second bioelectrical signal.

In some embodiments, each stacked electrode pair is arranged in an orthogonal, close, unipolar (OCU) configuration. As such, a bioelectrical signal received from a respective electrode pair comprises an OCU electrogram signal calculated by subtractive analysis. The subtractive analysis includes subtracting the second biological signal recorded by the second electrode from the first biological signal recorded by the first electrode.

In some embodiments, the plurality of electrode pairs are arranged in a nonlinear configuration and distributed across the array at known locations and each electrode pair is separated from one another by a known distance.

The bioelectrical signals may include, but are not limited to, electrocardiogram (ECG), electroencephalogram (EEG), electrocorticogram (ECoG or iEEG), electromyogram (EMG), electrooculogram (EOG), electroretinogram (ERG), electronystagmogram (ENG), electroolfactogram (EOG), electroantennogram (EAG), electrocochleogram (ECOG or ECochG), electrogastrogram (EGG), electrogastroenterogram (EGEG), electroglottogram (EGG), electropalatogram (EPG), electroarteriogram (EAG), electroblepharogram (EBG), electrodermogram (EDG), electrohysterogram (EHG), electroneuronogram (ENeG or ENoG), electropneumogram (EPG), electrospinogram (ESG), electrovomerogram (EVG), galvanic skin response (GSR), and electrodermal activity (EDA).

The target site generally comprises a tissue associated with an organ. The organ may include, but is not limited to, heart, brain, spinal cord, skeletal muscle, smooth muscle, eye, cochlea, stomach, bladder, bowel, and lungs. Accordingly, the tissue may include at least one of skin, muscle tissue, and nervous tissue. The tissue may be associated with one of the central nervous system (CNS) and peripheral nervous system (PNS).

The step of assessing a condition may include correlating the electrophysiological measurements with known electrophysiological measurements associated with the condition.

The method may further include a step of tracking at least a second indicator of the condition. The second indicator may include at least one of physiological function, psychological function, and cognitive function. As such, the assessment of the condition may further be based on the second indicator.

The method may further include the step of controlling delivery of an electrical stimulation therapy to the target site of the patient to treat the condition. The electrical stimulation is configured to treat one or more symptoms associated with the condition. The electrical stimulation may include, but is not limited to, deep brain stimulation (DBS), spinal cord stimulation (SCS), pelvic stimulation, gastric stimulation, peripheral nerve stimulation, and functional electrical stimulation of the target site.

In another embodiment, the invention provides an array of single (i.e., not stacked) electrodes arranged to facilitate measurement of electric potential in space over a tissue. For example, in cardiac tissue the array is positioned as a distribution of electrodes on the surface of the cardiac tissue so as to allow the computation of conduction velocity. In this embodiment, the electrodes are as described herein but without stacking and without the insulation layer. This simple embodiment allows for rapid computation of potentials across regions of a tissue.

### BRIEF DESCRIPTION OF THE DRAWINGS

FIG. 1 is a block diagram illustrating a system for monitoring bioelectrical activity and assessing conditions associated therewith.

FIG. 2 illustrates an electrode subsystem, including a catheter equipped with an electrode pair having an orthogonal, close, unipolar (OCU) electrode configuration.

FIG. 3 illustrates an example of improved spatial resolution obtained by use of an OCU electrode configuration.

FIG. 4 illustrates a two-dimensional multi-electrode array, in which each electrode includes an OCU electrode configuration.

FIG. 5 illustrates an exemplary common mode rejection (CMR) electrode configuration measuring a propagating bioelectrical signal associated with a tissue.

### DETAILED DESCRIPTION

By way of overview, the present invention provides systems and methods for monitoring bioelectrical activity of a desired target site via a unique and innovative electrode design. The target site may include any cell, tissue, and/or organ of a subject from which bioelectrical signals are to be collected and used for assessing a condition. The assessment of a condition may include diagnosing the subject with a particular disorder or disease based, at least in part, on the collected bioelectrical signals. Additionally, or alternatively, the assessment may include a determination of physiological, psychological, and cognitive effects as a result of application of a treatment, such as electrical stimulation or energy, or application of a medicament. Accordingly, the present invention is suitable for numerous applications in which bioelectrical activity monitoring is required.

For example, the present invention is useful for monitoring activity in a specific organ or system (i.e., heart, brain, nervous system, or the like), and such data collected can be used for assessing a condition (i.e., physiological, psychological, cognitive, etc.). By monitoring bioelectrical activity of a given target site, the collected signal data can be used in diagnosing a patient with a condition, including any known disorders, simply based on an analysis of the signal data. In turn, the present invention is further useful in monitoring bioelectrical activity of a given target site in response to application of treatment, such as application of electrical stimulation or energy. The present invention may also be useful in a research setting, including drug screening applications for testing the safety and efficacy of a drug on a subject (i.e., animal studies).

As will be described in greater detail herein, the unique electrode design includes a two-dimensional electrode array comprising either single electrodes or a plurality of stacked electrode pairs. In the paired electrode embodiment, each electrode pair is generally arranged in an orthogonal, close, unipolar (OCU) configuration. The OCU design provides the benefits of existing unipolar and bipolar electrode designs without the drawbacks, all while providing high spatial resolution, thereby improving bioelectrical signal sensing. As such, the present invention is suitable for numerous applications in which bioelectrical activity monitoring is required and high resolution is desired and is not limited to use in any given field.

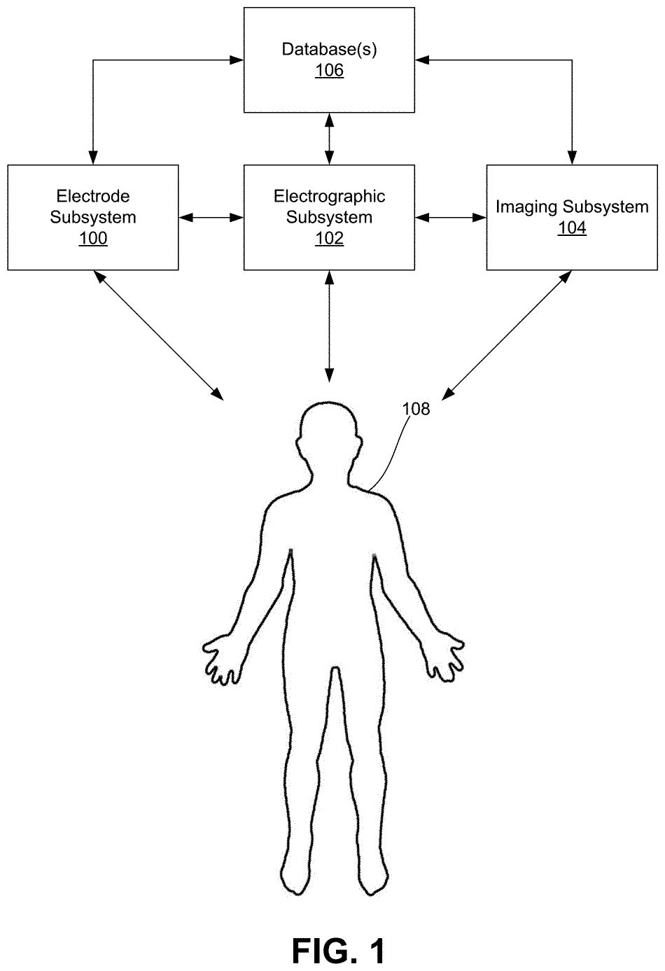

FIG. 1 is a block diagram illustrating a system for monitoring bioelectrical activity and assessing conditions associated therewith. The system includes an electrode subsystem 100, an electrographic subsystem 102, an imaging subsystem 104, and one or more databases 106 with which one or more of subsystems 100, 102, and 104 communicate and transmit data.

The electrode subsystem 100 may generally include a two-dimensional electrode array provided on a device. Accordingly, the electrode subsystem 100 may include one or more catheters (for supporting the electrode array). The electrode subsystem 100 may also include, but is not limited to, one or more surgical devices for accessing any particular patient 108 cell, tissue, or organ from which bioelectrical activity is to be monitored, one or more sheaths with one or more valves for preventing flowback, a saline solution for flushing components of the subsystem, one or more guidewires for positioning the one or more catheters, and/or one or more contrast agents (used in combination with an appropriate imaging 104 for viewing the target site during use. The electrode subsystem 100 may include a separate interface or display and/or share with other components of the system shown in FIG. 1. The electrode subsystem 100 and its components may be operated manually and/or automatically.

According to some embodiments of the present invention, the electrode subsystem 100 also may include, but is not limited to, one or more electrode localization technologies, such as triangulation-based localization, radio-frequency-based localization (e.g., the CARTO™ XP System, which is available from Biosense Webster® (Diamond Bar, Calif.)), and/or impedance-based localization (e.g., the EnSite NavX™ Navigation & Visualization Technology, which is available from St. Jude Medical (St. Paul, Minn.)).

The electrographic subsystem 102 is configured to collect electrophysiological measurements associated with the bioelectrical signal data recorded by the electrode array. Accordingly, the electrographic subsystem 102 may include, but is not limited to, one or more of the following electrographic modalities: electrocardiography, electroatriography, electroventriculography, intracardiac electrogram, electroencephalography, electrocorticography, electromyography, electrooculography, electroretinography, electronystagmography, electroolfactography, electroantennography, electrocochleography, electrogastrography, electrogastroenterography, electroglottography, electropalatography, electroarteriography, electroblepharography, electrodermography, electrohysterography, electroneuronography, electropneumography, electrospinography, electrovomerography. The electrographic subsystem 102 may include a separate display and/or share a display with other components of the system shown in FIG. 1.

The imaging subsystem 104 may be configured to acquire or collect measurements indicative of a tissue substrate's total boundary length, total surface area, and/or boundary-length-to-surface-area ratio. The imaging subsystem 104 may include any means by which a medical representation (e.g., a two-dimensional image or three-dimensional model) of a tissue substrate is acquired and/or generated. Suitable imaging modalities include, but are not limited to, MRI, CT, rotational angiography, three-dimensional ultrasound, and/or three-dimensional electro-anatomic mapping. Some imaging modalities may require the injection of one or more contrast agents. The imaging subsystem 104 may include a separate display and/or share a display with other components of the system shown in FIG. 1.

As previously described, the present invention allows for the monitoring of bioelectrical activity of a desired target site and further assessing a condition of a patient based on such bioelectrical activity. For example, the electrode subsystem 100, namely the two-dimensional electrode array (described in greater detail herein) is positioned at a target site of the patient 108. It should be noted that the patient may include any living specimen, and is not limited to a human as depicted. The electrode array comprises a plurality of stacked electrode pairs each comprising a first electrode configured to be in contact with a surface of the target site and a second electrode separated from the first electrode, wherein one or more the electrode pairs are arranged orthogonal to the surface of the target site.

The electrode array senses bioelectrical signals via one or more of the electrode pairs. In turn, electrophysiological measurements are obtained from the bioelectrical signals (via the electrographic subsystem 102) in response to bioelectrical activity associated with the target site. A condition of the patient can be assessed based, at least in part on, the electrophysiological measurements. The most common electrophysiological monitoring methods generally include Electroencephalography (EEG), Electromyography (EMG), and Electrocardiography (ECG). Electroencephalography (EEG) is an electrophysiological monitoring method to record electrical activity of the brain. It is typically non-invasive, with electrodes placed along the scalp, although invasive electrodes are sometimes used in specific applications. EEG measures voltage fluctuations resulting from ionic current within the neurons of the brain. Electromyography (EMG) is an electrophysiological monitoring process for evaluating and recording the electrical activity produced by skeletal muscles. EMG provides electrical feedback from voluntary muscle functions and, with external electrical stimulation responses, provides feedback about neuro-muscular functionality. Normal or abnormal nerve conduction can be detected by stimulating nerves with electrical pulse and simultaneously measuring the delay in which get motoric unit potential from another point of the body with a known distance. Electrocardiography (ECG) is the process of recording the electrical activity of the heart over a period of time using electrodes placed on the skin. These electrodes detect the tiny electrical changes on the skin that arise from the heart muscle's electrophysiologic pattern of depolarizing and repolarizing during each heartbeat. It should be noted, however, that the present invention may utilize any known electrophysiological measurement methods and modalities.

The condition may include at least one of a physiological disorder, psychological disorder, and cognitive disorder. The condition may include, for example, Alzheimer's disease, Parkinson's disease, other types of movement disorders, seizure disorders (e.g., epilepsy), urinary or fecal incontinence, sexual dysfunction, obesity, mood disorders, gastroparesis, or diabetes. Accordingly, the target site may include a tissue associated with an organ selected from the group consisting of a heart, brain, spinal cord, skeletal muscle, smooth muscle, eye, cochlea, stomach, bladder, bowel, and lungs. The tissue may include at least one of skin, muscle tissue, and nervous tissue. In the instance of Parkinson's disease, for example, the tissue is associated with one of the central nervous system (CNS) and peripheral nervous system (PNS), which may include brain tissue. The electrode array may be placed in direct contact with the brain tissue so as to collect bioelectrical signal data.

In order to assess the condition, the specific electrophysiological measurements may be correlated with known electrophysiological measurements associated with the condition. In other words, certain conditions may have a known bioelectrical signal profile indicative of the condition. As such, the measuring electrophysiological measurements may be compared with bioelectrical signal profiles stored (in databases 106) and, upon a positive correlation, the condition can be identified. Yet still, in addition to relying on the electrophysiological measurements for assessing a condition, the systems may further rely on a second indicator for assessing the condition. The second indicator may include at least one of physiological function, psychological function, and cognitive function. For example, in the instance of Parkinson's disease, the secondary indicator may be a measurement of movement (i.e., tremors) of the patient, including whether the tremors have decreased post application of a medicament (i.e., during drug trial) or application of stimulation. In the instance of a cognitive disorder (i.e., Alzheimer's disease), the secondary indicator may be a measurement of the patient's performance on a cognitive test, including whether their performance has decreased or improved post application of a medicament (i.e., during drug trial) or application of stimulation.

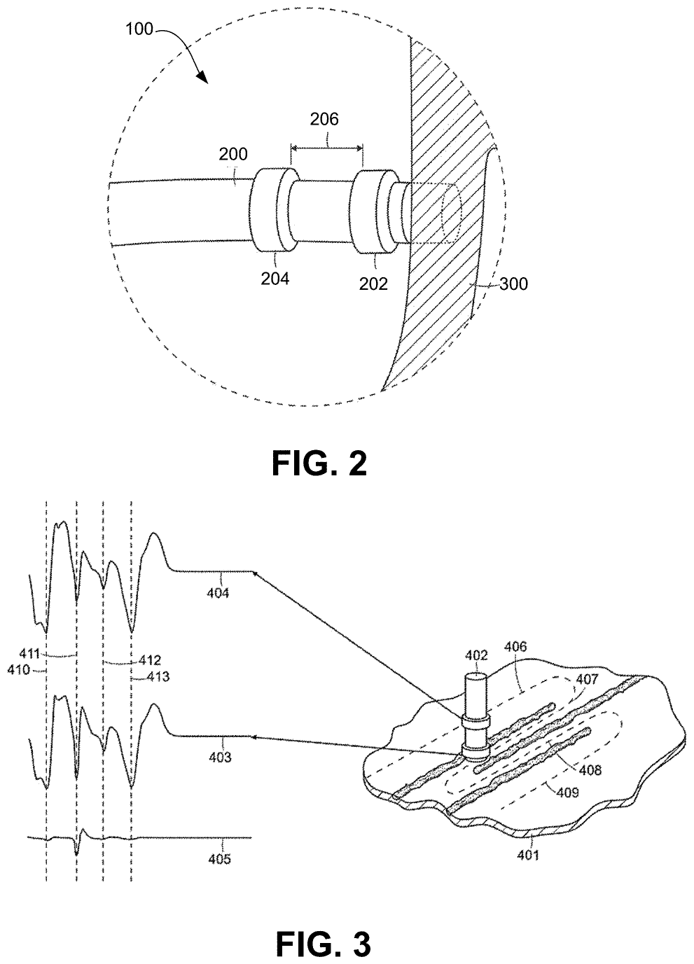

FIG. 2 illustrates an electrode subsystem 100, including a catheter equipped with an electrode pair having an orthogonal, close, unipolar (OCU) electrode configuration. As shown, the electrode design consists of a two-dimensional electrode array comprising at least one stacked electrode pair (first electrode 202 and second electrode 204 provided on a catheter 200). The first electrode 202 (also referred to as the “index electrode”) is configured to be in contact with a surface of a desired target site 300 (i.e., cells, tissue, organ or the like from which bioelectrical signals are to be recorded) and the second electrode 204 (also referred to as the “indifferent electrode”) is separated from the first electrode 202. The electrode pair is generally arranged in an orthogonal, close, unipolar (OCU) configuration. More specifically, the common axis between the first and second electrodes (referred to as the “inter-electrode axis”) is “orthogonal” to a given surface at the target site when a recording is performed. The distance 206 between the first and second electrodes (referred to as “inter-electrode distance”) is substantially “close”, which may be within an order of magnate of the electrode size (i.e., from 0.1 mm to 3.0) or greater, such that the second electrode is “close” enough to the surface of the target site to detect a bioelectrical signal. The electrode pair may be “unipolar” in that only the first electrode may be in contact with the surface of the target site.

Such a configuration addresses the limitations of existing unipolar and bipolar electrodes. In particular, recorded electrical potential of current bipolar electrodes vary with their orientation relative to the direction of a passing wavefront. Additionally, because bipolar electrodes have both electrodes on a given surface, there is potential inclusion of distinctly different electrical activity from each electrode. As such, by providing electrodes oriented perpendicular to the tissue plane (i.e., via the orthogonal close unipolar (OCU) design of the present invention), the two-dimensional electrode array of the present disclosure retains the superior near/far-field discrimination of common bipolar electrode recordings with the directional independence and smaller footprint of unipolar recordings. Furthermore, the unipolar electrode configuration of the present invention retains all of the spatial resolution benefits of a contact bipolar configuration, but with the additional spatial resolution enhancement conferred by a smaller footprint (i.e., only half of the electrodes, the first electrode of each electrode pair, may be in contact with the tissue surface).

Accordingly, the unique configuration of the electrode array (i.e., the OCU design) provides the benefits of existing unipolar and bipolar electrode designs without the drawbacks, all while providing high spatial resolution, thereby improving bioelectrical signal sensing. As such, the present invention is suitable for numerous applications in which bioelectrical activity monitoring is required and high resolution is desired, and is not limited to use in any given field.

FIG. 3 illustrates an example of improved spatial resolution obtained by use of an OCU electrode configuration on cardiac tissue. The tissue substrate surface 401 is assessed by a catheter with one pair of recording electrodes in an OCU electrode configuration 402. The index electrode records electrogram signal 403, and the indifferent electrode records electrogram signal 404. The resulting OCU electrogram signal 405 is calculated by subtracting the indifferent electrogram 404 from the index electrogram 403. As shown, the tissue substrate surface 401 contains three linear non-conducting scars (resulting, e.g., from ablation lesions). The scars separate the tissue surface 401 into four conducting channels 406-409. The index electrode of the catheter is in close proximity to and/or touching the tissue surface 401 directly above the second conducting channel 407. As indicated by the dashed arrows, the path of tissue activation within the vicinity of the recording electrodes is serpentine. The index electrode and indifferent electrode electrogram signals 403-404 exhibit large deflections at times 410-413 as the tissue activation wavefront moves through the conducting channels 406-409. However, the OCU electrogram signal x05 exhibits very small deflections at times 410 and 412-413 as the tissue activation wavefront moves through the conducting channels 406 and 408-409 not directly beneath the catheter, and a much larger deflection at time 411 as the tissue activation wavefront moves through the local conducting channel 407 directly beneath the catheter. Also, while each cell in the tissue surface 401 was activated only once, the index electrode and indifferent electrode electrograms 403-404 feature four deflections, indicating that a measurement of the frequency content of either electrogram would be higher than the true tissue activation frequency content of tissue surface 401. Meanwhile, the frequency content of the OCU electrogram 405 is a more likely indicator of the true tissue activation frequency.

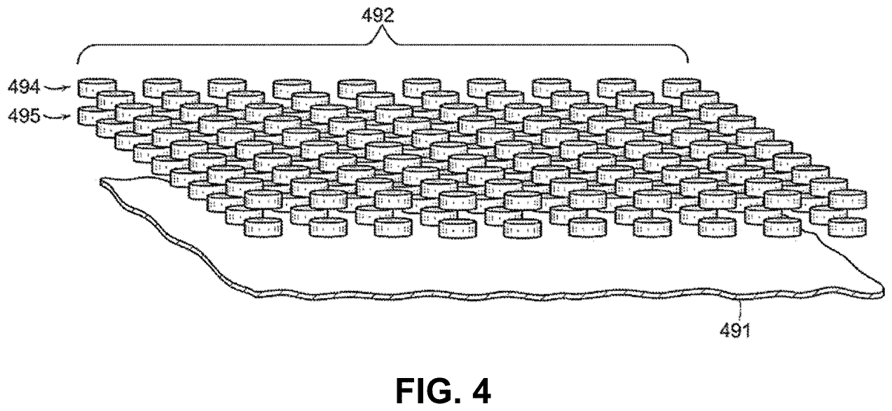

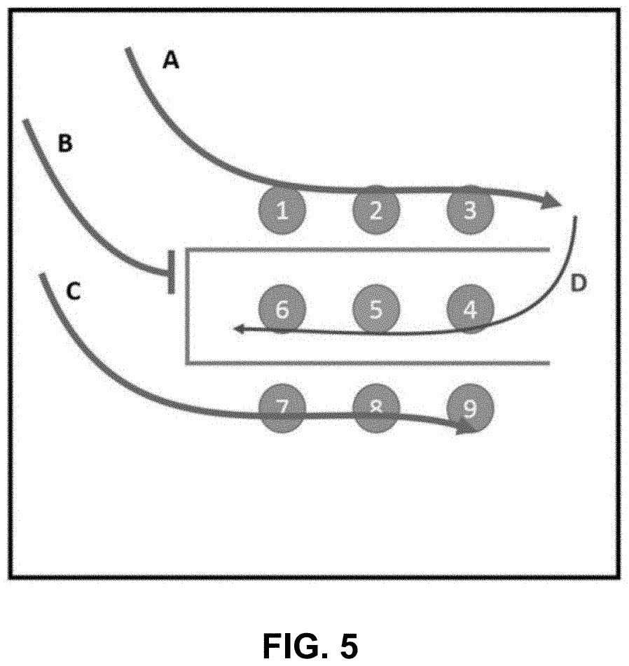

FIG. 4 illustrates a two-dimensional multi-electrode array, in which each electrode includes an OCU electrode configuration. As shown, the two-dimensional multi-electrode array 492 has been deployed over a tissue substrate surface 491. In this example, a total count of 100 index electrodes 493 are paired with a total count of 100 indifferent electrodes 494, and each pair of electrodes is itself in OCU configuration. It should be noted that any amount of electrode pairs may be provided within an array.

As previously described, in another embodiment, the invention provides a two-dimensional array of single electrodes arranged to facilitate measurement of electric potential in space over a tissue. Such a configuration is referred to herein as a common mode rejection (CMR) electrode configuration.

FIG. 5 illustrates an exemplary CMR electrode array of the present disclosure. As illustrated, as opposed to being arranged in a stacked configuration (i.e., pairs of electrodes), the CMR array is comprised of single electrodes, which may include microelectrodes, distributed across the array at known locations and each electrode is separated by a known distance. This simple embodiment allows for rapid computation of potentials across regions of a tissue. For example, electrodes in the CMR array may be useful in detecting signals of interest at bioelectrical signals propagate through tissue underneath the CMR array.

A “central” electrode in the array that detects the local activation signal works in conjunction with multiple “surrounding” contact electrodes that surround the central electrode on the array. As a local signal passes underneath the central electrode, a signal is concurrently recorded from both the central electrode and the surrounding electrodes. The signals from the surrounding electrodes are averaged, and the resulting average is subtracted from the central electrode signal.

As a result, the CMR electrode array is able to accurately measure a given signal (e.g., an activation signal when used in cardiac tissue) by eliminating both far-field signal interference and near-field signal interreference from other electrodes on the array. As a result, a signal detected by the central electrode represents only the signal generated by the activation signal as it passes underneath the electrode. The CMR electrode array is thus able to leverage the benefits of unipolar electrodes and bipolar electrodes, while eliminating their drawbacks.

Accordingly, the use of simultaneously obtained electrode data according to the invention enables one to determine relative positions of measurements made by multiple electrodes in the construction of tissue mapping and the like (e.g., cardiac mapping).

For example, the CMR may be useful in cardiac applications, wherein the CMR array may be positioned as a distribution of electrodes on the surface of the cardiac tissue so as to allow the computation of conduction velocity. The array can be used to construct a map of cardiac rhythm and tissue properties, such as scarring.

Referring to FIG. 5, the wave of a local activation signal (A, B, and C) travels from the top left to the bottom right of the figure across a tissue on which the array is placed. When the wave encounters, for example, an open-ended scar (shown as a grey three-sided rectangular shape), which is a common feature in arrythmias, the wave is cannot propagate straight through the scar. Such scars are known to not conduct excitation energy. Thus, the wave must propagate around the scar. As shown, the wave (D) propagates around the scar and into the region that the open-ended scar surrounds.

As shown, the electrodes of the array are positioned both within and outside the scar region. The array of electrodes comprises a series of nine electrodes, labeled 1-9, and arranged in three rows. However, other numbers and arrangements of electrodes are contemplated by the invention, for example, concentric circles, spirals, etc. The central electrode, electrode 5, is colored orange. The surrounding electrodes, electrodes 1-4 and 6-9, are colored blue.

As such, the use of simultaneously obtained electrode data according to the invention enables one to determine relative positions of measurements made by multiple electrodes in the construction of a map of cardiac rhythm. Methods of the invention, by which direction of activation is projected to the heart surface, are unaffected by motion (e.g., cardiac or respiratory). This, then, allows the generation of a more precise cardiac map and avoids the impact that motion has on projections of non-simultaneously acquired electrode data onto the cardiac surface. In such methods, as the activation signal propagates in the tissue underneath the array, the signal is measured by a series of consecutive central electrodes. Thus, as the wave propagates, an electrode that may have been a surrounding electrode is tasked as a “new” central electrode. Concurrently, appropriate electrodes are tasked as “new” surrounding electrodes for the new central electrode.

While the CMR electrode array is described as being useful in cardiac-related monitoring and mapping (e.g., construct a map of cardiac rhythm and tissue properties), the CMR electrode array is useful for monitoring bioelectrical activity of a desired target site may include any cell, tissue, and/or organ of a subject from which bioelectrical signals are to be collected and used for assessing a condition and/or mapping function and or objects.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for assessing a condition of a patient, the method comprising:
positioning a two-dimensional electrode array at a target site, the electrode array comprising a plurality of stacked electrode pairs each comprising a first electrode configured to be in contact with a surface of the target site and a second electrode separated from the first electrode, wherein one or more the electrode pairs are arranged orthogonal to the surface of the target site;
sensing bioelectrical signals via one or more of the electrode pairs;
obtaining electrophysiological measurements from the bioelectrical signals in response to bioelectrical activity associated with the target site; and
assessing a condition of the patient based, at least in part on, the electrophysiological measurements.

2. The method of claim 1, wherein the first electrode of each stacked electrode pair is configured to record a first bioelectrical signal and the second electrode of each stacked electrode pair is separated from the first electrode by a distance which enables the second electrode to record a second bioelectrical signal.

3. The method of claim 2, wherein each stacked electrode pair is arranged in an orthogonal, close, unipolar (OCU) configuration.

4. The method of claim 3, wherein a bioelectrical signal received from a respective electrode pair comprises an OCU electrogram signal calculated by subtractive analysis.

5. The method of claim 4, wherein the subtractive analysis comprises subtracting the second biological signal recorded by the second electrode from the first biological signal recorded by the first electrode.

6. The method of claim 2, wherein the plurality of electrode pairs are arranged in a nonlinear configuration and distributed across the array at known locations and each electrode pair is separated from one another by a known distance.

7. The method of claim 1, wherein the bioelectrical signals are selected from the group consisting of electrocardiogram (ECG), electroencephalogram (EEG), electrocorticogram (ECoG or iEEG), electromyogram (EMG), electrooculogram (EOG), electroretinogram (ERG), electronystagmogram (ENG), electroolfactogram (EOG), electroantennogram (EAG), electrocochleogram (ECOG or ECochG), electrogastrogram (EGG), el ectrogastroenterogram (EGEG), electroglottogram (EGG), electropalatogram (EPG), electroarteriogram (EAG), electroblepharogram (EBG), electrodermogram (EDG), electrohysterogram (EHG), electroneuronogram (ENeG or ENoG), electropneumogram (EPG), electrospinogram (ESG), electrovomerogram (EVG), galvanic skin response (GSR), and electrodermal activity (EDA).

8. The method of claim 1, wherein the target site comprises a tissue associated with an organ.

9. The method of claim 8, wherein the organ is selected from the group consisting of a heart, brain, spinal cord, skeletal muscle, smooth muscle, eye, cochlea, stomach, bladder, bowel, and lungs.

10. The method of claim 9, wherein the tissue comprises at least one of skin, muscle tissue, and nervous tissue.

11. The method of claim 10, wherein the tissue is associated with one of the central nervous system (CNS) and peripheral nervous system (PNS).

12. The method of claim 1, wherein assessing a condition comprises correlating the electrophysiological measurements with known electrophysiological measurements associated with the condition.

13. The method of claim 1, further comprising tracking at least a second indicator of the condition.

14. The method of claim 13, wherein the second indicator comprises at least one of physiological function, psychological function, and cognitive function.

15. The method of claim 14, wherein assessment of the condition is further based on the second indicator.

16. The method of claim 1, wherein the condition comprises at least one of a physiological disorder, psychological disorder, and cognitive disorder.

17. The method of claim 1, further comprising controlling delivery of an electrical stimulation therapy to the target site of the patient to treat the condition.

18. The method of claim 17, wherein electrical stimulation is selected from the group consisting of deep brain stimulation (DBS), spinal cord stimulation (SCS), pelvic stimulation, gastric stimulation, peripheral nerve stimulation, and functional electrical stimulation of the target site

19. The method of claim 18, wherein the electrical stimulation is configured to treat one or more symptoms associated with the condition.

20. The method of claim 19, wherein the condition comprises at least one of Alzheimer's disease, Parkinson's disease, other types of movement disorders, seizure disorders (e.g., epilepsy), urinary or fecal incontinence, sexual dysfunction, obesity, mood disorders, gastroparesis, or diabetes.
